(19) United States
(12) Patent Application Publication
(10) Pub. No.: US 2023/0022547 A1
(43) Pub. Date: Jan. 26, 2023

(54) Urinary Detection and Collection Systems and Methods

(71) Applicant: C. R. Bard, Inc., Franklin Lakes, NJ (US)

(72) Inventors: Younggeun Cho, Covington, GA (US); Samatha Gaddam, Suwanee, GA (US); Puja Patel, Lawrenceville, GA (US); Danielle Garner, Snellville, GA (US); Madieyna Ndiaye, Athens, GA (US); Sharanya Jangiti, Buford, GA (US)

(21) Appl. No.: 17/870,698

(22) Filed: Jul. 21, 2022

Related U.S. Application Data
(60) Provisional application No. 63/225,285, filed on Jul. 23, 2021.

(51) Int. Cl. A61B 5/20; A61B 5/00; A61B 5/145

(52) U.S. Cl. CPC A61B 5/208; A61B 5/6802; A61B 5/0022; A61B 5/746; A61B 5/6808; A61B 5/14507

(57) ABSTRACT

Disclosed herein is a urinary detection system including a plurality of sensors configured to detect a urine sample and one or more analytes within the urine sample, the plurality of sensors selected from the group consisting of a moisture sensor, a pH sensor, a blood sensor, a bacteria sensor, and combinations thereof. The system further includes a console in communication with the plurality of sensors, the console including one or more processors, an energy source, non-transitory computer readable medium and a plurality of logic modules.

### PRIORITY

This application claims the benefit of priority to U.S. Provisional Application No. 63/225,285, filed Jul. 23, 2021, which is incorporated by reference in its entirety into this application

### BACKGROUND

Locally capturing and analyzing urine can be divided into two separate events. Locally capturing urine can require either a urine hat deployed in a bathroom setting or a urine catheter connected to a urine collection bag. Analyzing the captured urine can require a patient to transport the captured urine to a laboratory environment for analysis, where the patient and their clinician must then wait for the results before any type of treatment can begin. It would be beneficial to the patient and the clinician to be able to quickly and locally detect and analyze urine including at the point of capture for faster analysis. Disclosed herein is a system and method of use that address the foregoing.

### SUMMARY

Disclosed herein is a urine detection system that according to some embodiments, includes a number of sensors including first sensors configured to detect urine excreted by a patient and second sensors configured to measure a number of analytes of the urine. The system further includes a console in communication with the number of sensors, where the console includes an energy source, a number of processors, and memory including a non-transitory computer readable medium having logic stored thereon that, when executed by the processors, performs operations of the system. The operations include detecting the urine excreted by the patient and obtaining analyte measurements of the urine.

In some embodiments of the urine detection system, the first sensors include a moisture sensor, the second sensors include one or more of a pH sensor, a blood sensor, or a bacteria sensor, and the analytes include one or more of iron, hemoglobin, or bacteria.

In some embodiments of the urine detection system, the console includes a wireless module, and at least a subset of the number of sensors is wirelessly coupled with the console.

In some embodiments of the urine detection system, the operations further include transmitting the detecting the urine to a computing device.

In some embodiments of the urine detection system, the operations further include obtaining analyte measurements of the urine in response to the detecting the urine and transmitting the analyte measurements to the computing device.

In some embodiments of the urine detection system, the computing device is coupled with electronic medical record system.

Also disclosed herein is a urine collection system that, according to some embodiments, includes an external urinary catheter and a urine collection bag fluidly coupled with the external urinary catheter via a drainage tube of the external urinary catheter. The system further includes a urine detection system that includes a number of sensors including first sensors configured to detect the presence of urine excreted by a user and second sensors configured to obtain analyte measurements of the urine. The system further includes a console in communication with the sensors, where the console includes an energy source, a number of processors, and memory including a non-transitory computer readable medium having logic stored thereon that, when executed by the processors, performs operations of the system, and where the operations include (i) detecting the urine and (ii) obtaining the analyte measurements of the urine.

In some embodiments of the urine collection system, the drainage tube is detachably coupled with at least one of the external urinary catheter or the collection bag.

In some embodiments, the urine collection system further includes a diaper coupled with the external urinary catheter, where the diaper includes a liquid absorbent layer and a liquid impermeable layer disposed external to the liquid absorbent layer, and where one or more of the first sensors are coupled with the liquid absorbent layer.

In some embodiments of the urine collection system, one or more of the first sensors are coupled with the drainage tube.

In some embodiments, the urine collection system further includes a collection bag holder configured for containing the collection bag, where the collection bag holder has a strap configured to secure the collection bag holder to the user.

In some embodiments, the urine collection system further includes a drainage pump coupled in line with the drainage tube, where the pump is configured to displace urine along the drainage tube to the collection bag. In such embodiments, the drainage pump is coupled with the console and the operations further include activating the drainage pump in response to detecting the urine.

In some embodiments of the urine collection system, the console includes a wireless module, and the operations further include (i) defining a correlated a time of day with the detecting the urine and (ii) wirelessly transmitting the detecting the urine and the correlated time of day to a computing device.

In some embodiments of the urine collection system, the operations further include obtaining the analyte measurements in response to the detecting the urine and transmitting the analyte measurements to the computing device.

In some embodiments of the urine collection system, the operations further include (i) comparing the analyte measurements with one or more analyte limits stored in memory; and (ii) as a result of the comparison, generating one or more alerts when any of the analyte measurements are outside of the one or more analyte limits; and (iii) transmitting the one or more alerts to the computing device.

In some embodiments of the urine collection system, the first sensors include a first drainage tube sensor disposed upstream adjacent the pump and a second drainage tube sensor disposed downstream adjacent the pump, and the operations further include (i) determining a count of pump cycles when both the first and second drainage tube sensors detect urine within the drainage tube and (ii) calculating a volume of urine delivered by the pump to the urine collection bag based the count of pump cycles.

Also disclosed herein is a method of collecting, by a urine collection system, urine excreted by a user, that includes (i) detecting the urine excreted by the user via one or more first sensors of the urine collection system (ii) obtaining one or more analyte measurements of the urine via one or more first sensors of the urine collection system, (iii) capturing the urine within a urine collection container of the system; and (iv) transmitting one or more analyte measurements to a computing device.

In some embodiments of the method, the one or more analytes include a pH, hemoglobin, iron, or bacteria.

In some embodiments of the method, the urine collection system includes an external urinary catheter coupled with a diaper, where the diaper includes a liquid absorption layer and a liquid impermeable layer disposed external the liquid absorption layer, and where the liquid impermeable layer is configured to funnel the urine toward the external urinary catheter. In such embodiments, the urine collection system further includes a pump coupled in line with a drainage tube of the external urinary catheter, where the drainage tube extends to the urine collection container. In such embodiments, the method further includes pumping urine along the drainage tube from the diaper to the collection container.

In some embodiments of the method, the first sensors include a first drainage tube sensor disposed upstream adjacent the pump and a second drainage tube sensor disposed downstream adjacent the pump, and the method further includes (i) determining a count of pump cycles when both the first and second drainage tube sensors detect urine within the drainage tube and (ii) calculating a volume of urine delivered by the pump to the urine collection container based the count of pump cycles.

These and other features of the concepts provided herein will become more apparent to those of skill in the art in view of the accompanying drawings and following description, which describe particular embodiments of such concepts in greater detail.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a block diagram of some components of a urinary detection system including a console, in accordance with some embodiments;

FIG. 2 illustrates a perspective view of the urinary detection system used with a urine collection system, in accordance with some embodiments;

FIG. 3 illustrates a block diagram of some components of a urine collection system that includes the urinary detection system and, in accordance with some embodiments;

FIGS. 4A-4C illustrates a perspective view of an exemplary method of detecting a urine and measuring one or more analytes within the urine using the urine collection system, in accordance with some embodiments;

FIG. 5 illustrates a flow chart of an exemplary method of detecting a urine and measuring one or more analytes within the urine, in accordance with some embodiments;

FIG. 6 is a detailed illustration of the pump and related components of the system of FIG. 2, in accordance with some embodiments; and

FIG. 7 is a flow chart illustrating a method for determining a volume of the urine collected by the system of FIG. 2, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

Unless defined otherwise, all technical and scientific terms used herein have the same meaning as commonly understood by those of ordinary skill in the art.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

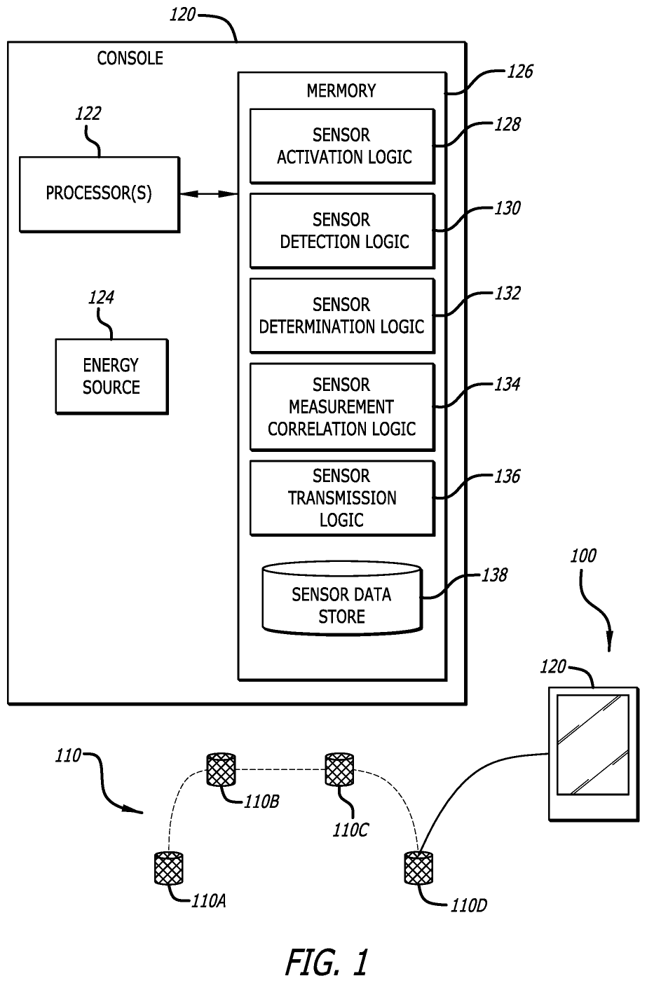

FIG. 1 illustrates a block diagram of some components of a urinary detection system 100 including a console 120, in accordance with some embodiments. The urinary detection system (“system”) 100 includes a number of sensors 110 each in communication with the console 120. At least a subset of the sensors 110 are configured to detect the presence of a liquid (e.g., urine). At least a subset of the sensors 110 are configured to measure (including detect) various analytes within the liquid, and transmit the measurement of the various analytes to the console 120. In some embodiments, the console 120 may be configured to transmit the analyte measurement to a computing device (including a cell phone), an electronic medical record (“EMR”) system or the like. In some embodiments, the system 100 may be in communication other medical devices or systems, and logic of the system 100 may be configured to activate other medical devices or systems in response to the measurement of one or more analytes of the urine.

The sensors 110 may include moisture sensors 110A configured to detect the presence of a liquid in the location the sensors 110A, pH sensors 110B configured to measure a pH of the liquid, blood sensors 110C configured to detect the presence of blood (e.g., components of blood such as hemoglobin) within the liquid, or bacteria sensors 110D configured to detect the presence of bacteria within the liquid. In some embodiments, the pH sensors 110B may include pH strips configured to change color corresponding to the pH of the liquid. In some embodiments. the pH sensors 110B may include an electronic pH meter. In some embodiments, the blood sensors 110C may include (i) hemoglobin sensors configured to detect the presence of hemoglobin in the liquid, (ii) iron sensors configured to detect the presence of iron in the liquid or other sensors that may detect additional components of blood in the liquid. In some embodiments, the sensors 110 may be configured to detect the presence of proteins (e.g., albumin, creatinine or the like) or the presence of sugars in the liquid. In some embodiments, the sensors 110 may be configured to detect the presence of one or more ions or compounds (e.g., sodium, potassium, calcium, magnesium, manganese, nickel, ammonium, uric acid, urea or the like) within the liquid. In some embodiments, detecting one or more ions or compounds within the liquid may allow logic of the system 100 to identify the liquid as urine versus sweat, water, or some other liquid. In some embodiments, one or more of the sensors 110 may include electrical sensors, biosensors or a combination thereof. The system 100 may be deployed in various situations the capture and/or analysis of urine may be advantageous, such as a hospital or other healthcare environment. For example, the system 100 may be deployed on a hospital bed to detect when a patient excretes urine. By way of another example, the system 100 may be deployed in an ambulatory environment to detect when ambulatory patient or a user generally excretes urine within a urine collection system as described herein below.

In some embodiments, one or more of the sensors 110 may be configured for a single use (e.g., disposable). Any or all of the sensors may be coupled with the console 120 via a wired connection or a wireless connection. Exemplary wireless communication modalities can include WiFi, Bluetooth, Near Field Communications (NFC), cellular Global System for Mobile Communication (“GSM”), electromagnetic (EM), radio frequency (RF), combinations thereof, or the like. In some embodiments, the console 120 may include one or more processors 122, an energy (electrical power) source 124, a non-transitory computer readable medium (“memory”) 126, and logic which may conclude various logic modules. In some embodiments, the energy source 124 may include a battery which may be a rechargeable battery. In some embodiments, the energy source 124 may be detachably coupled with the console 120. In some embodiments, the battery may be recharged via a wired connection with a charger or via induction. The energy source 124 may be configured to provide power to each the sensors 110. In other embodiments, one or more of the sensors 110 may be self-powered. In some embodiments, energy source 124 may continuously provide power to one or more of the sensors 110, such as the moisture sensors 110A so that the system 100 may continuously monitor for the presence of liquid, for example.

In the illustrated embodiment, the logic modules include a sensor activation logic 128, a sensor detection logic 130, a sensor determination logic 132, a sensor correlation logic 134, sensor transmission logic 136, and a data store 138. The activation logic 128 may be configured to activate one or more of the sensors 110. In some embodiments, the activation logic 128 may be configured to activate one or more of the sensors 110 independently or all of the sensors 110 simultaneously. In an embodiment, the activation logic 128 may activate one or more sensors 110 in response to input from the user, such as via an actuator (e.g., a button, a switch, or the like) coupled to the console 120.

The sensor detection logic 130 may be configured to obtain/receive information/data from the sensors 110 and/or perform operations, in accordance with the information/data, according to some embodiments. The sensor detection logic 130 may obtain information/data related to the detection of a liquid (e.g., urine). The detection logic 130 may also obtain information/data pertaining to the measurement of analytes within the liquid. In some embodiments, the sensor detection logic 130 may obtain the information/data from the different sensors 110 sequentially or simultaneously. For example, sensor detection logic 130 may first obtain information/data the moisture sensor 110A pertaining to the detection of liquid, and thereafter, the sensor detection logic 130 may obtain information/data pertaining to measurements of analytes from the sensors 110B-110D.

The sensor determination logic 132 may be configured process the information/data obtained by the sensor detection logic 130, in accordance with the information/data, according to some embodiments. In the illustrated embodiment, the sensor determination logic 132 may convert the information/data into specific measurements of the analytes. For example, the sensor determination logic 132 may convert the information/data obtained from the pH sensor 110B into an actual pH number (e.g., 7). In some embodiments, the sensor determination logic 132 may be configured to compare the measurements of the analytes with limits and/or ranges for the analytes stored in the memory 126 to determine if the measurements are within or exceed the limits and/or ranges. In some embodiments, the sensor determination logic 132 may identify the liquid as urine (i.e., determine that the liquid is urine or is not urine) based on the comparison. In some embodiments, the limits and/or ranges may be standardized, or pre-determined. In some embodiments, the limits and/or ranges may be adjusted by the clinician to take in account situations where excessive analytes concentrations may reside within the urine. For example, if a patient has recently undergone a medical procedure, the clinician may anticipate a lower than normal pH in the urine and may lower a pH range to account for the lower than normal pH values. In some embodiments, the sensor determination logic 132 may generate one or more alerts, when any of the measurements fall outside of the limits and/or ranges.

The sensor correlation logic 134 may be configured to correlate the information/data with a time of day at which the information/data was obtained, in accordance with some embodiments. In other words, the sensor correlation logic 134 may assign a time stamp with the information/data. For example, the sensor correlation logic 134 may assign a time stamp to information/data pertaining to the detection of liquid, so that the system 100 knows the time of day that the patient excreted urine. By way of another example, the sensor correlation logic 134 may assign a time stamp to information/data pertaining to the analyte measurements, so that the system 100 knows the time of day that the analytes at the measured values were present in the excreted urine.

The sensor transmission logic 136 may be configured to wirelessly transmit information to an external computing device, in accordance with some embodiments. The external computing device may be a stand-alone device such as personal computer, tablet or a cell phone, or external computing device may be part of a system such as an electronic medical record (EMR) system. The information may include one or more of the detection of a liquid, or the analyte measurements, any of each of which may include time stamps. The information may further include one or more alerts. In some embodiments, the sensor transmission logic 136 may transmit the information according to a regular schedule, such as every 30 minutes, for example. In some embodiments, the sensor transmission logic 136 may transmit the information immediately upon the occurrence of an event, such as the detection of the liquid or the generation of an alert, for example.

In some embodiments, the data store 138 may be configured to store all or any portion of all information/data obtained or generated by the logic modules, according to some embodiments. For example, the data store 138 may store the liquid detection events, analyte measurements, results of the comparisons, time stamps, or transmitted information/notifications.

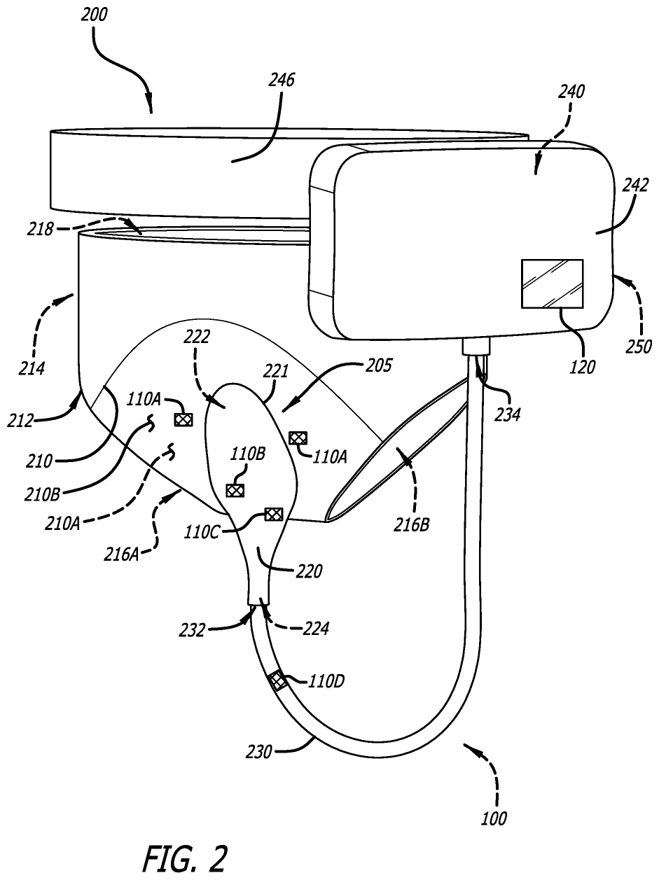

FIG. 2 illustrates a perspective view of a urine collection system 200 that includes or otherwise employs the urinary detection system 100 described above, in accordance with some embodiments. The urine collection system 200 may include all or a subset of the components and functionalities of the system 100. Generally speaking, the urine collection system 200 may facilitate the collection of urine from a user, and the included system 100 may detect the urine and measure analytes of the urine during collection or immediately thereafter (e.g., within a few minutes).

The urine collection system 200 generally includes an external catheter 205 (catheter) 205 (i.e., a male urinary catheter or a female urinary catheter) coupled with a drainage tube 230 that extends to a urine collection bag (bag) 240.

The urine collection system 200 further includes an liquid absorption device 210 (e.g., a diaper) configured to be worn around the pelvic/groin region of an ambulatory or non-ambulatory user. In some embodiments, the urine absorption device 210 may be coupled with the catheter 205 so that the urine absorption device 210 secures the position of the catheter 205. In some embodiments, the catheter 205 may be configured to receive urine from the patient's urethra directly. In other embodiments, the urine absorption device 210 may define a portion (e.g., a funnel portion) of the catheter 205. The urine absorption device 210 may be configured to absorb urine excreted by the user. The urine absorption device 210 may also be configured to wick away the urine from the skin surface of the patient. In some embodiments, the urine absorption device 210 may be configured to receive and capture therein the urine excreted by the user. The bag 240 may be secured in a urine collection bag holder 242 that may include to a strap 246 configured to secure the bag 240 to the user. The strap 246 may be configured to wrap around a portion of the body of the user (e.g., a torso, an appendage or the like) and position the bag 240.

The urine absorption device 210 may include a front side 212, a back side 214, two leg apertures 216A-216B and a torso aperture 218. The urine absorption device 210 may include a device (e.g., hook and loop fastener, elastic material around the leg apertures 216A-216B and torso aperture 218, a belt, or the like) for securing the urine absorption device 210 to the user. The urine absorption device 210 may be manufactured in single size and during deployment, each user can adjust the urine absorption device 210 for a proper fit. The patient may place one leg into the leg aperture 216A, another leg into the leg aperture 216B and slide the urine absorption device 210 up onto the torso and then use the securing device to secure the urine absorption device 210 to the user. The urine absorption device 210 may include a number (e.g., 1, 2, 3 or more) of absorption layers 210A configured to absorb urine and wick the urine away from the skin surface of the user the one or more layers configured to absorb urine and wick the urine away from the skin surface of the user. The urine absorption device 210 may further include a liquid impermeable layer 201B disposed exterior to the absorption layers 210A, where the liquid impermeable layer 201B layer is configured to prevent urine from leaking out of the absorption layers 210A. In some embodiments, the pH sensors 110B include pH strips, and the pH strips may be coupled with or integrated into the absorption layers 210A. In the illustrated embodiment, the urine absorption device 210 is configured for single use.

The catheter 205 may include a urine funneling device 220 configured to funnel the urine sample from the urine absorption device 210 into the drainage tube 230, as such the funneling device 220 combined with the drainage tube 230 may define the catheter 205. The urine funneling device 220 may be integrated into the urine absorption device 210 or the urine absorption device 210 may include a urine funneling device aperture 221 configured to allow the urine funneling device 220 be threaded therethrough. The urine funneling device 220 may be coupled to the front side 212, allowing the user to excrete a urine through the urine funneling device aperture 221 into the urine funneling device 220. The urine funneling device 220 may include a user opening 222 in fluid communication with a drainage opening 224. The user opening 222 may be larger than the drainage opening 224, the user opening 222 configured to collect the urine excreted by the user and direct the urine to the drainage opening 224. The drainage opening 224 may be coupled to a first end 232 of a drainage tube 230, and a second end 234 of the drainage tube 230 may be coupled to bag 240 to define fluid communication between the urine funneling device 220 and the urine collection bag 240.

The bag 240 is configured to contain the urine excreted by the user. In some embodiments, the urine may flow passively through drainage tube 230 due to gravity. In other embodiments, the system 200 may include a pump 250 to displace urine along the drainage tube 230 to the bag 240. The pump 250 may be positioned adjacent the bag 240. The pump 250 may be configured to create a negative pressure so as to draw the urine away from the urine absorption device 210 toward the bag 240. The pump 250 may be coupled with the bag 240 or integrated into the bag 240. The urine funneling device 220 may be detachably coupled to the drainage tube 230 or detachably coupled to the urine absorption device 210, allowing the urine absorption device 210 or the urine funneling device 220 to be disposable. In some embodiments, one or more the urine funneling device 220, the drainage tube 230, the urine collection bag 240, the console 120, or the pump 250 may be configured for single use.

The sensors 110 may be deployed in various configurations and various locations within the urine collection system 200. For example, one or more of the sensors 110 may be located within the urine absorption device 210, the urine funneling device 220, the drainage tube 230, or the bag 240. In some embodiments, one or more of the moisture sensors 110A may be coupled to the urine absorption device 210 to detect the presence of urine at a first point of contact while the pH sensor 110B, the blood sensor 110C, and the bacteria sensor 110D may be located within the urine funneling device 220, the drainage tube 230 or the urine collection bag 240 so that the pH sensor 110B, the blood sensor 110C, and the bacteria sensor 110D may contact (have access to) an adequate volume of the urine. The console 120 may be coupled to the urine collection bag 240, coupled to the pump 250 or integrated into the urine collecting bag holder 242. In an embodiment where one or more sensors 110 are wired to the console 120, the one or more sensors 110 may be disconnectable from the console 120.

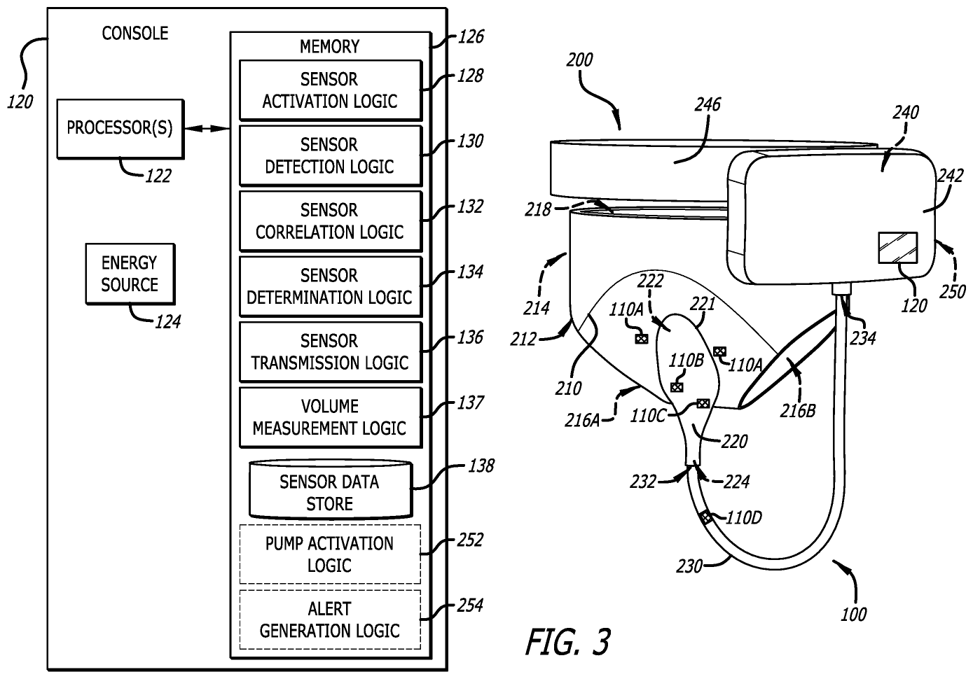

FIG. 3 illustrates a block diagram of some components of the urinary detection system 100 used with the urine collection system 200. In some embodiments, the console 120 includes the one or more processors 122, the energy source 124, the non-transitory computer readable medium (“memory”) 126 and the plurality of logic modules. In some embodiments, the plurality of logic modules may include one or more of the sensor activation logic 128, the sensor detection logic 130, the sensor correlation logic 134, the sensor determination logic 132, the sensor transmission logic 136, a volume measurement logic 137, a sensor data store 138, a pump activation logic 252, and an alert generation logic 254. The sensor activation logic 128 may include a moisture sensor activation logic, a pH sensor activation logic, a blood sensor activation logic, and a bacteria sensor activation logic. The sensor detection logic 130 may include a moisture sensor detection logic, a pH sensor detection logic, a blood sensor detection logic, and a bacteria sensor detection logic. In some embodiments, the moisture sensor detection logic may be configured to activate one or more of the sensors 110B-110C and one or more other logic modules. For example, the moisture sensor 110A may be continuously activated to detect the presence of the urine. If the presence of the urine is detected, the other sensors 110B-110C may be activated to measure the analytes. In some embodiments, the sensor determination logic 132 may include a moisture sensor determination logic, a pH sensor determination logic, a blood sensor determination logic, and a bacteria sensor determination logic. In some embodiments, the sensor determination logic 132 may compare the measurements of analytes with established limits and/or ranges for each sensor to determine if the measurements are within or exceed the limits and/or ranges.

In some embodiments, the sensor correlation logic 134 and the sensor transmission logic 136 may be configured to work as described above. In some embodiments, the logic modules may further include the pump activation logic 252, and the alert generation logic 254. When the urinary detection system 100 is used with the urine collection system 200 and the urine collection system 200 includes the pump 250 coupled to the urine collection bag 240, the pump activation logic 252 may be configured to activate the pump 250 to draw urine into the bag 240. In some embodiments, the moisture sensor determination logic may be configured to activate the pump activation logic 252. In some embodiments, the alert generation logic 254 may be configured to generate an alert in various scenarios including when the moisture sensor detects the presence of urine, when the one or more analytes are above the established threshold, or when the urine within the urine collection bag 240 is approaching or has reached the maximum capacity of the urine collection bag 240.

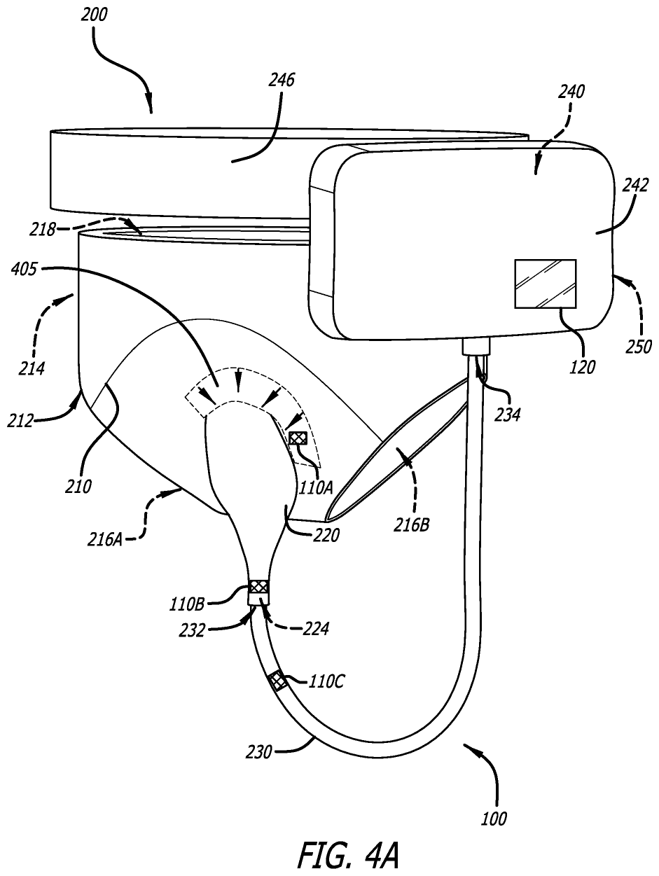

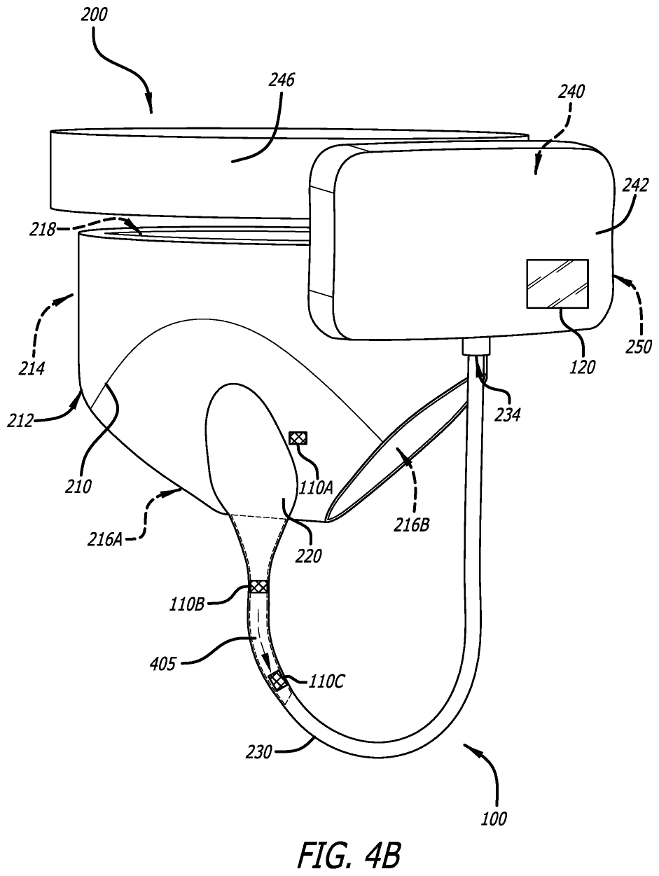

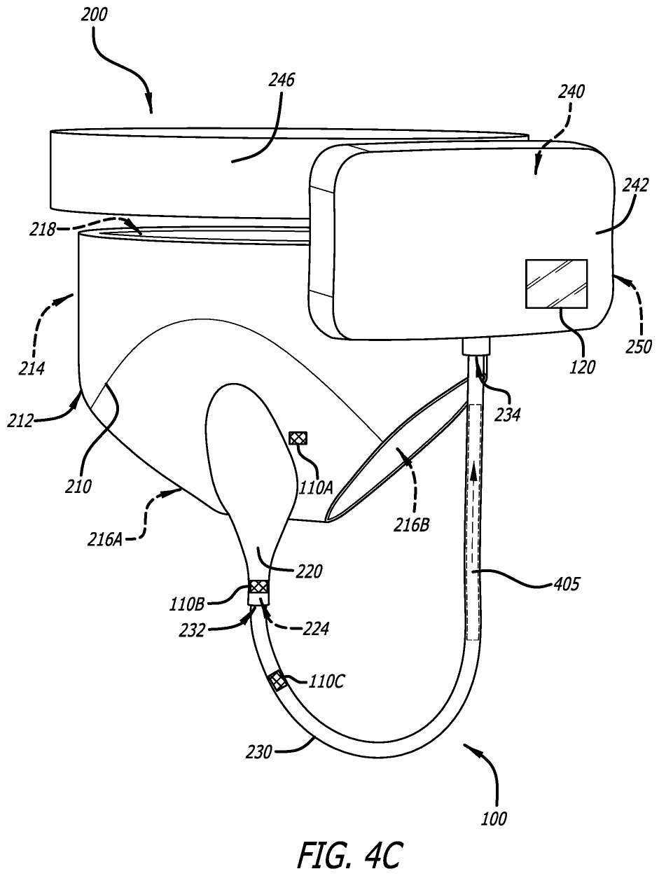

FIGS. 4A-4C illustrates perspective views the system 200 depicting an exemplary method 300 of detecting a urine sample 405 and obtaining measurements of analytes within a urine sample 405 using the urinary detection system 100 include by the urine collection system 200, in accordance with some embodiments. As illustrated in FIG. 4A, the urinary detection system 100 and the urine collection system 200 are as described above. A user may excrete a urine sample 405 into the urine absorption device 210. The one or more moisture sensors 110A may be configured to detect the urine sample 405 and communicate to the console 120 the detection of the urine sample 405. In some embodiments, the console 120 may communicate to the computing device of the detection of a urine sample 405. In some embodiments, the one or more moisture sensors 110A may be continuously activated for detecting additional urine samples. In some embodiments, the detection of the urine sample 405 initiate the activation of the sensors 110B-110C. The urine absorption device 210 may be configured to direct the urine sample 405 into the urine funneling device 220. In some embodiments, the combination of the urine absorption device 210 and gravity flow may be configured to direct the urine sample 405 into the urine funneling device 220.

In FIG. 4B, the pump 250 is activated by the logic to create a negative pressure environment within the drainage tube 230 to draw the urine sample 405 into the drainage tube 230. As the urine sample 405 moves from the urine funneling device 220 into the drainage tube 230, the pH sensor 110B and the blood sensor 110C may detect and measure one or more analytes within the urine sample. The pH sensor 110B and the blood sensor 110C may communicate to the console 120 the detection and measurements (e.g., concentrations) of the one or more analytes within the urine sample 405.

As illustrated in FIG. 4C, the urine sample 405 may travel through the drainage tube 230 to reach the urine collection bag 240. In some embodiments, the pH sensor 110B and the blood sensor 110C may alternatively be located within the urine collection bag 240 so as to detect and measure the one or more analytes of the urine sample 405 within the urine collection bag 240. The logic may obtain the measurements of the analytes from the pH sensor 110B and the blood sensor 110C console 120 and may communicate the measurement to the computing device.

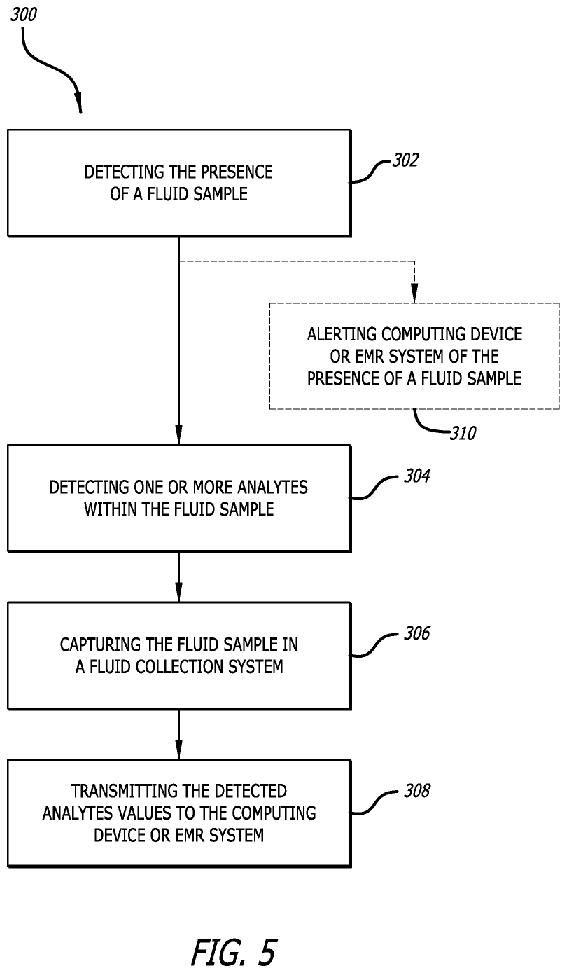

FIG. 5 illustrates a flow chart of the exemplary method 300 of detecting a urine sample and one or more analytes within the urine sample, in accordance with some embodiments. In some embodiments, the method 300 includes detecting the urine sample (block 302). In some embodiments, detecting the urine sample includes the moisture sensors in communication with the console detecting the urine sample. In some embodiments, detecting the urine sample includes continuously activating the moisture sensors to detect the urine sample. In some embodiments, the method 300 may include communicating an alert to the computing device pertaining to the detection of the urine sample (block 310). In some embodiments, communicating an alert to the computing device include wirelessly transmitting the alert to the computing device.

In some embodiments, the method 300 includes measuring one or more analytes within the urine sample (block 304). In some embodiments, detecting one or more analytes within the urine sample includes the sensors measuring the one or more analytes within the urine sample. In some embodiments, the one or more analytes may include analytes, such as pH, blood components, protein, ions, or bacteria for example. In some embodiments, measuring the one or more analytes within the urine sample include comparing the measurements with one or more limits/ranges for the respective analytes stored in memory.

In some embodiments, the method 300 includes activating the pump of the urine collection system 200 (block 3) to draw the urine sample into the urine collection bag 240. In some embodiments, the method 300 includes transmitting information to the computing device (block 4), such as the detection of urine, and/or the measurements of analytes, any of which may include time stamps. The information may further include one or more alerts. In some embodiments, transmitting the information to the computing device includes wirelessly transmitting the detected analyte values to the computing device.

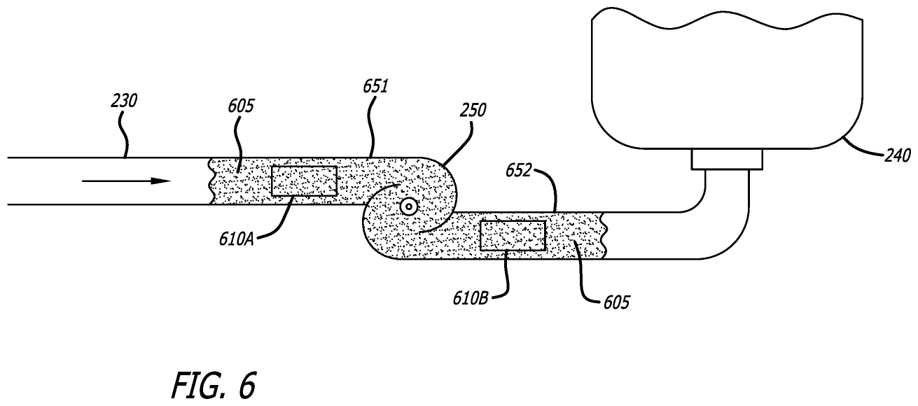

FIG. 6 is a detailed illustration of the pump 250 and associated components. FIG. 6 depicts the drainage tube 230 coupled with an inlet 651 of the pump 250 and the bag 240 coupled with an outlet 652 of the pump 250. A urine sample 605 extends between the inlet 651 and the outlet 652. A first moisture sensor 610A is operatively coupled with the drainage tube 230 adjacent the inlet 651 so that the moisture sensor 610A may detect the presence of the urine sample 605 within the inlet 651. A second moisture sensor 610B is operatively coupled with the drainage tube 230 adjacent the outlet 652 so that the moisture sensor 610B may detect the presence of the urine sample 605 within the outlet 652. The moisture sensors 610A, 610B may in some respects resemble the moisture sensors 110A.

The pump 250 may be a positive displacement pump, such as a piston pump or a vane pump, for example. As such, the pump 250 may transport a defined quantity of urine with each cycle (e.g., revolution) of the pump 250 as long as the pump 250 is primed with urine between the inlet 651 and the outlet 652. Therefore, the volume of urine delivered to the bag 240 may be determined by counting the cycles of the pump 250 while the pump 250 is primed with urine. The volume of urine delivered to the bag 240 by the pump 250 may be directly related to the volume excreted by the user.

The first and second moisture sensors 610A, 610B may be utilized to determine the priming status of the pump 250. FIG. 6 depicts a first instance, where the first moisture sensor 610A and the second moisture sensor 610B both detect the presence of urine within the drainage tube 230. As such in the first instance, logic may deduce than the pump 250 is primed with urine, i.e., that urine extends between the first moisture sensor 610A and the second moisture sensor 610B. In an alternative second instance (not shown), either the first moisture sensor 610A, the second moisture sensor 610B, or both do not detect urine. As such, in the second instance, the pump 250 is not primed with urine.

The volume measurement logic 137 may be configured to determine the volume of urine excreted by the user. In use, the sensor detection logic 130 may activate the pump 250 when urine is detected by the moisture sensors 110A to transport urine to the bag 240. In some embodiments, the volume measurement logic 137 may activate the first and second moisture sensors 610A, 610B in response to activation of the pump 250. When first and second moisture sensors 610A, 610B both detect the presence of urine, the volume measurement logic 137 may initiate counting the cycles of the pump 250. When either the first moisture sensor 610A or the second moisture sensors 610A, 610B detect the absence of the urine, the volume measurement logic 137 may discontinue counting the cycles of the pump 250. The volume measurement logic 137 may then multiple the number cycles counted by a defined quantity of urine per cycle of the pump 250 to determine the volume of urine delivered to the bag 240 which may estimate the volume of urine excreted by the user.

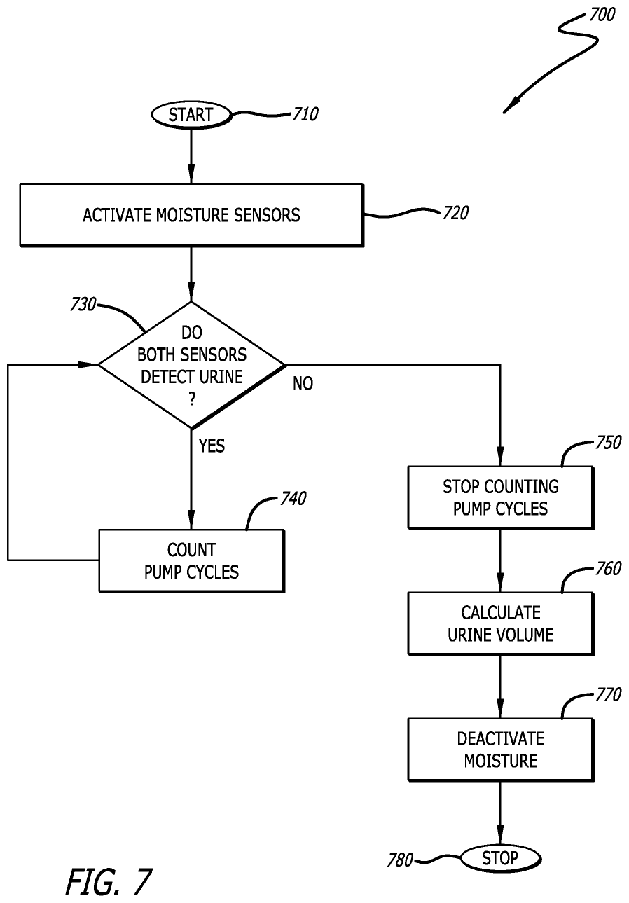

FIG. 7 is a flow chart illustrating an exemplary method for determining a volume of urine excreted by the user and collected by the system 200, according to some embodiments. The description that follows makes reference to FIG. 6. The method 700 includes all or a subset of the following steps or processes. The state of the system 200 at the start (block 710) of the process includes the system 200 operatively coupled with the user, the user has excreted urine, the pump 250 is activated response to urine detected by at least one moisture sensor 110A. A step of the method 700 includes activating the first and second moisture sensors 610A, 610B (block 720) to monitor for the presence of urine adjacent the pump 250. Another step includes checking to see if both the first and second moisture sensors 610A, 610B detect urine (block 730) meaning that the pump 250 is primed with urine. If so, the next step is to count the cycles of the pump (block 740) and then continue checking to see if both the first and second moisture sensors 610A, 610B, detect urine, i.e., return to block (block 730). In the case that either of the first or second moisture sensors 610A, 610B detect the absence of urine (i.e., do not detect urine) meaning the pump 250 is not primed, the next step includes discontinuing the counting of pump cycles (block 750). A next step may include calculating the volume of urine excreted by the user (block 760) which may include summing up the counted pump cycles and multiplying the total number of pump cycles by the defined quantity per cycle of the pump 250 to determine the volume of urine delivered to the bag 240 by the pump 250 (block 760). The method 700 may further include deactivating the first and second moisture sensors 610A, 610B (block 770) before stopping the method (block 780). The method 700 may be repeated during use of the system 200 to measure additional volumes of the urine excreted by the user.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A urine detection system, comprising:
a number of sensors including:
first sensors configured to detect urine excreted by a patient; and
second sensors configured to measure a number of analytes of the urine;
and
a console in communication with the number of sensors, the console comprising an energy source, a number of processors, and memory including a non-transitory computer readable medium having logic stored thereon that, when executed by the processors, performs operations of the system, including:
detecting the urine excreted by the patient; and
obtaining analyte measurements of the urine.

2. The system according to claim 1, wherein:
the first sensors include a moisture sensor,
the second sensors include one or more of a pH sensor, a blood sensor, or a bacteria sensor, and
the analytes include one or more of iron, hemoglobin, or bacteria.

3. The system according to claim 1, wherein:
the console includes a wireless module, and
at least a subset of the number of sensors are wirelessly coupled with the console.

4. The system according to claim 3, wherein the operations further include transmitting the detecting the urine to a computing device.

5. The system according to claim 4, the operations further include:
obtaining analyte measurements of the urine in response to the detecting the urine; and
transmitting the analyte measurements to the computing device.

6. The system according to claim 5, wherein the computing device is coupled with electronic medical record system.

7. A urine collection system, comprising:
an external urinary catheter;
a urine collection bag fluidly coupled with the external urinary catheter via a drainage tube of the external urinary catheter; and
a urine detection system, comprising:
a number of sensors including:
first sensors configured to detect the presence of urine excreted by a user; and
second sensors configured to obtain analyte measurements of the urine; and
a console in communication with the sensors, the console comprising an energy source, a number of processors, and memory including a non-transitory computer readable medium having logic stored thereon that, when executed by the processors, performs operations of the system, including:
detecting the urine; and
obtaining the analyte measurements of the urine.

8. The system according to claim 7, wherein the drainage tube is detachably coupled with at least one of the external urinary catheter or the collection bag.

9. The system according to claim 7, further comprising:
a diaper coupled with the external urinary catheter, wherein:
the diaper includes:
a liquid absorbent layer; and
a liquid impermeable layer disposed external to the liquid absorbent layer, and
one or more of the first sensors are coupled with the liquid absorbent layer.

10. The system according to claim 7, wherein one or more of the first sensors are coupled with the drainage tube.

11. The system according to claim 7, further comprising:
a collection bag holder configured for containing the collection bag, the collection bag holder having a strap configured to secure the collection bag holder to the user.

12. The system according to claim 7, further comprising a pump coupled in line with the drainage tube, the pump configured to displace urine along the drainage tube to the collection bag, wherein:
the pump is coupled with the console, and
the operations further include activating the pump in response to the detecting the urine.

13. The system according to claim 7, wherein:
the console includes a wireless module, and
the operations further include:
defining a correlated a time of day with the detecting the urine; and
wirelessly transmitting the detecting the urine and the correlated time of day to a computing device.

14. The system according to claim 7, wherein the operations further include:
obtaining the analyte measurements in response to the detecting the urine; and
transmitting the analyte measurements to the computing device.

15. The system according to claim 7, wherein the operations further include:
comparing the analyte measurements with one or more analyte limits stored in memory;
as a result of the comparison, generating one or more alerts when any of the analyte measurements are outside of the one or more analyte limits; and
transmitting the one or more alerts to the computing device.

16. The system according to claim 7, wherein;
the first sensors include:
a first drainage tube sensor disposed upstream adjacent the pump; and
a second drainage tube sensor disposed downstream adjacent the pump, and the operations further include:
determining a count of pump cycles when both the first and second drainage tube sensors detect urine within the drainage tube; and
calculating a volume of urine delivered by the pump to the urine collection bag based the count of pump cycles.

17. A method of collecting, by a urine collection system, urine excreted by a user, comprising:
detecting the urine excreted by the user via one or more first sensors of the urine collection system;
obtaining one or more analyte measurements of the urine via one or more first sensors of the urine collection system;
capturing the urine within a urine collection container of the system; and
transmitting one or more analyte measurements to a computing device.

18. The method according to claim 17, wherein the one or more analytes include a pH, hemoglobin, iron, bacteria, protein or sugar.

19. The method according to claim 17, wherein the urine collection system includes:
an external urinary catheter coupled with a diaper, the diaper including a liquid absorption layer and a liquid impermeable layer disposed external the liquid absorption layer, the liquid impermeable layer configured to funnel the urine toward the external urinary catheter; and
a pump coupled in line with a drainage tube of the external urinary catheter, the drainage tube extending to the urine collection container, the method further comprising:
pumping urine along the drainage tube from the diaper to the collection container.

20. The method according to claim 17, wherein the first sensors include:
a first drainage tube sensor disposed upstream adjacent the pump; and
a second drainage tube sensor disposed downstream adjacent the pump, the method further comprising:
determining a count of pump cycles when both the first and second drainage tube sensors detect urine within the drainage tube; and
calculating a volume of urine delivered by the pump to the urine collection container based the count of pump cycles.
